(19) United States
(12) Patent Application Publication
(10) Pub. No.: US 2022/0202666 A1
(43) Pub. Date: Jun. 30, 2022

(54) ORAL CARE COMPOSITION

(71) Applicant: BASF SE, Ludwigshafen (DE)

(72) Inventors: Jeniree Andreina Flores Delgado, Tarrytown, NY (US); Denis Ontumi, Tarrytown, NY (US); Anna Anita Gripp, Florham Park, NJ (US); Marc De Mul, Tarrytown, NY (US); Leonora Ethleen Leigh, Tarrytown, NY (US)

(21) Appl. No.: 17/594,625

(22) PCT Filed: Apr. 22, 2020
(86) PCT No.: PCT/US2020/029263

§ 371 Date: Oct. 25, 2021

Related U.S. Application Data
(60) Provisional application No. 62/838,967, filed on Apr. 26, 2019.

(51) Int. Cl. A61K 8/26; A61Q 11/00

(52) U.S. Cl. CPC A61K 8/26; A61K 2800/87; A61K 2800/28; A61Q 11/00

(57) ABSTRACT

Disclosed herein are an oral care composition comprising a magnesium aluminum phyllosilicate abrasive with a high pellicle cleaning ratio (PCR) and a low relative dentin abrasivity (RDA), a method of using said oral care composition, method of its preparation, and a kit encompassing it.

### RELATED APPLICATIONS

This application claims priority to U.S. Provisional Patent Application No. 62/838,967, filed Apr. 26, 2019, which is herein incorporated by reference in its entirety.

### FIELD OF THE INVENTION

The present invention relates to the field of oral care compositions, in particular, to dentifrice compositions exhibiting high pellicle cleaning ratio and low dentin abrasion, methods of preparation thereof, methods of use thereof, and kits encompassing the composition.

### BACKGROUND OF THE INVENTION

Most dentifrice compositions contain different types of silica to aid in the cleaning of the mouth. The silica acts as an abrasive that can help remove stains from teeth, and in doing so, it can erode the surface of the enamel, leaving consumers exposed to experiencing sensitivity. Another use for silica in the toothpaste formulation is as a thickening agent that can help deliver desired rheological properties. However, one type of silica only is not capable of delivering both benefits—cleaning and thickening—therefore, formulators usually use two different types of silica in the same formulation to obtain the desired product and benefits.

There is a need to provide a product that can clean as well as current silica products do, but that is gentler and less abrasive to the enamel, and that can also thicken the formulation to help achieve the desired rheological properties, preferably with only one product.

### OBJECTS AND SUMMARY OF THE INVENTION

The present disclosure aims at providing an oral care composition that is natural, cleans surfaces in the oral cavity well, minimizes harm to surfaces in the oral cavity, is less abrasive to enamel, has favorable rheological properties, and can be composed of fewer components since one or more of its components have multiple functionalities.

The above objects of the present invention and others may be achieved by the present disclosure which in certain embodiments is directed to an oral care composition, a method of preparing an oral care composition, a method of cleaning a surface and/or treating a condition in an oral cavity of a subject with an oral care composition, and/or a kit including an oral care composition. Said oral care composition may be in the form of a toothpaste, gel, powder, mouth wash, mousse, tablet, and combinations thereof.

The oral care composition disclosed herein may comprise magnesium aluminum phyllosilicate and an excipient. The magnesium aluminum phyllosilicate may act as an abrasive (having a high pellicle cleaning ratio (PCR)) and as a thickening agent (contributing to the rheological properties of the oral care composition) while being a naturally occurring composite that is gentle on the surface being cleaned (having a low relative dentin abrasivity (RDA)).

The oral care composition may have one or more of the following properties: 1) comprise an abrasive that has PCR:RDA ratio of about 0.9 to about 10, about 1.2 to about 5, or about 1.5 to about 3; and/or 2) a pH of about 6 to about 9.5, about 7 to about 9, or about 8 to about 9; and/or 3) a viscosity of about 25,000 cps to about 350,000 cps, from about 100,000 cps to about 300,000 cps, or about 150,000 cps to about 275,000 cps.

In some embodiments, the present disclosure is directed to a method of preparing any of the oral care compositions disclosed herein by combining magnesium aluminum phyllosilicate and an excipient.

In some embodiments, the present disclosure is directed to a method of cleaning a surface (e.g., teeth, gums, tongue) and/or treating a condition in an oral cavity of a subject by contacting the surface (e.g., brushing, rinsing) of a subject in need thereof with any of the oral care compositions disclosed herein.

In some embodiments, the present disclosure is directed to a kit comprising any of the oral care compositions disclosed herein enclosed in a suitable container (e.g., a toothpaste tube, a mouth wash bottle, a pressurizable container). The kit may further comprise one or more of toothbrush, rinsing cup, floss, use instructions, and combinations thereof.

### BRIEF DESCRIPTION OF THE DRAWINGS

The above and other features of the present disclosure, their nature, and various advantages will become more apparent upon consideration of the following detailed description, taken in conjunction with the accompanying drawings, in which:

FIG. 1 depicts a plot of the viscosity at different concentrations of abrasive in a toothpaste formulation.

FIG. 2 depicts pellicle cleaning ratio (PCR) and relative dentin abrasivity (RDA) values of various abrasive samples.

FIG. 3 depicts the relative dentin abrasivity (RDA) values of formulations spanning across a range of abrasive concentrations for an abrasive according to an embodiment as compared to high cleaning silica.

FIG. 4 depicts the pellicle cleaning ratio (PCR) values of formulations spanning across a range of abrasive concentrations for an abrasive according to an embodiment as compared to high cleaning silica.

### DEFINITIONS

As used herein, the singular forms “a,” “an,” and “the” include plural references unless the context clearly indicates otherwise. Thus, for example, reference to “an abrasive” includes a single abrasive as well as a mixture of two or more different abrasives, and the like.

As used herein, the term “about” in connection with a measured quantity, refers to the normal variations in that measured quantity, as expected by one of ordinary skill in the art in making the measurement and exercising a level of care commensurate with the objective of measurement and the precision of the measuring equipment. In certain embodiments, the term “about” includes the recited number ±10%, such that “about 10” would include from 9 to 11.

As used herein, the terms “active agent” and “active ingredient” refer to any material that is intended to produce a therapeutic, prophylactic, or other intended effect, whether or not approved by a government agency for that purpose. These terms with respect to specific agents include all active agents, all pharmaceutically acceptable salts thereof, complexes, crystalline forms, co-crystals, ether, esters, hydrates, solvates, and mixtures thereof.

The term “patient” refers to a subject, an animal or a human, who has presented a clinical manifestation of a particular symptom or symptoms suggesting the need for treatment, who is treated preventatively or prophylactically for a condition, or who has been diagnosed with a condition to be treated. The term “subject” is inclusive of the definition of the term “patient” and does not exclude individuals who are otherwise healthy.

“Pharmaceutically acceptable salts” include, but are not limited to, inorganic acid salts such as hydrochloride, hydrobromide, sulfate, phosphate and the like; organic acid salts such as formate, acetate, trifluoroacetate, maleate, tartrate and the like; sulfonates such as methanesulfonate, benzenesulfonate, p-toluenesulfonate and the like; amino acid salts such as arginate, asparaginate, glutamate and the like; metal salts such as sodium salt, potassium salt, cesium salt and the like; alkaline earth metals such as calcium salt, magnesium salt and the like; and organic amine salts such as triethylamine salt, pyridine salt, picoline salt, ethanolamine salt, triethanolamine salt, discyclohexylamine salt, N,N′-dibenzylethylenediamine salt and the like.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to illuminate certain materials and methods and does not pose a limitation on scope. No language in the specification should be construed as indicating any non-claimed element as essential to the practice of the disclosed materials and methods.

The term “condition” or “conditions” refers to those medical conditions that can be treated or prevented by administration to a subject of an effective amount of an active agent. Exemplary medical conditions may include, without limitations, caries, gingivitis, hypersensitivity, plaque formation, calculus formation, tooth stains, halitosis, combinations thereof, and the like.

The terms “treatment of” and “treating” includes the lessening of the severity of or cessation of a condition or lessening the severity of or cessation of symptoms of a condition, e.g., an oral condition such as plaque.

The terms “prevention of” and “preventing” includes the avoidance of the onset of a condition, e.g., an oral condition such as plaque.

“Therapeutically effective amount” is intended to include an amount of an active agent, or an amount of the combination of active agents, e.g., to treat or prevent the condition, or to treat the symptoms of the condition, in a subject.

The phrase “pharmaceutically acceptable” refers to those compounds, materials, and/or compositions, within the scope of sound medical judgment, suitable for use in contact with the tissues of human beings and animals without excessive toxicity, irritation, allergic response, or other problems or complications commensurate with a reasonable benefit/risk ratio.

### DETAILED DESCRIPTION

This disclosure is directed in certain embodiments to a natural product that can provide comparable or better cleaning of the mouth than conventional products with silica abrasives, but that is gentler and less abrasive to the enamel. The material that is used in oral care compositions disclosed herein may replace silica by acting as an abrasive and may also act as a thickening agent in the composition. In certain embodiments, the present disclosure incorporates magnesium aluminum phyllosilicate (e.g., attapulgite clay) in an oral care composition (e.g., a toothpaste formulation). The magnesium aluminum phyllosilicate may have multiple functionalities, such as: 1) delivering proper cleaning of the mouth, 2) being gentle on the enamel, and 3) thickening the formulation to obtain an advantageous rheology for the oral care composition (e.g., dentifrice).

In certain embodiments, the instant disclosure may be directed to a method of thickening (e.g., adjusting the viscosity) any of the oral care compositions described herein by incorporating a magnesium aluminum phyllosilicate into the oral care composition.

In certain embodiments, the magnesium aluminum phyllosilicate may be useful as a colorant due to its tan color. In certain embodiments, the instant disclosure may be directed to a method of coloring any of the oral care compositions described herein (e.g., paste, mouthwash, and so on) by incorporating a magnesium aluminum phyllosilicate into the oral care composition.

There are many advantages for incorporating magnesium aluminum phyllosilicate into the oral care compositions disclosed herein. Since the oral care compositions disclosed herein clean the mouth while being less abrasive, they can help prevent and/or reduce erosion of the enamel and the likelihood of a consumer experiencing sensitivity or alleviating its effect.

Additionally, utilizing a single component (i.e., the magnesium aluminum phyllosilicate) which contributes in a variety of aspects to the final properties of the oral care composition reduces the number of components that formulators need to use. Thus, formulators can forego using multiple products to attain certain rheological properties or cleaning benefits in the final oral care composition since the cleaning and thickening properties of the oral care composition may be fulfilled with the incorporation of the single magnesium aluminum phyllosilicate component.

According to various embodiments, the present disclosure is related to an oral care composition comprising a magnesium aluminum phyllosilicate and an excipient.

The magnesium aluminum phyllosilicate in the oral care compositions discussed herein has a multiple functions to act as an abrasive (i.e., provide gentle cleaning with minimal harm to the enamel) and as a thickener to enhance the viscosity of the oral care composition. In some embodiment, the magnesium aluminum phyllosilicate might also be used to color the oral care composition (e.g., a toothpaste).

Magnesium aluminum phyllosilicate may be represented, in some embodiments, by the chemical formula (Mg,Al)₂Si₄O₁₀(OH).4(H₂O). In certain embodiments, the magnesium aluminum phyllosilicate may be an attapulgite clay.

In some embodiments, the magnesium aluminum phyllosilicate may be a composite of smectite and palygorskite. The smectite may be expanding lattice clays, whereas palygorskite may have an acicular bristle-like crystalline form that does not swell or expand.

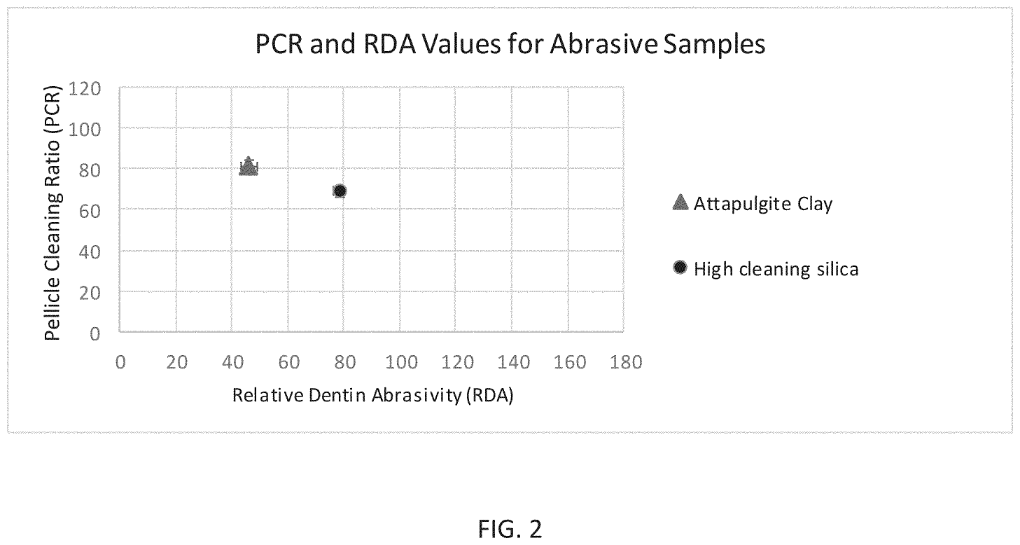

The oral care composition may comprise magnesium aluminum phyllosilicate (e.g., attapulgite clay), which may have a pellicle cleaning ratio value (PCR) of from about 60 to about 100, from about 65 to about 95, or from about 70 to about 90. The PCR value may be calculated as detailed with reference to FIG. 2 in Example 3. A higher PCR value is indicative a better cleaning performance (i.e., a greater amount of stained pellicle removed).

The oral care composition may comprise magnesium aluminum phyllosilicate (e.g., attapulgite clay), which may have a relative dentin abrasivity value (RDA) of from about 20 to about 70, from about 25 to about 65, or from about 35 to about 55. The RDA value may be calculated as detailed with reference to FIG. 2 in Example 4. A higher RDA value is indicative of greater relative dentin abrasivity (i.e., greater harm to the enamel of the tooth).

The ratio of PCR:RDA of the magnesium aluminum phyllosilicate (e.g., attapulgite clay) that may be incorporated in the oral care compositions disclosed herein may be from about 0.9 to about 10, from about 1.2 to about 5, or from about 1.5 to about 3. A higher PCR:RDA ratio may be more advantageous as it may be indicative that the final oral care composition would exhibit better cleaning performance while being gentler on the surface that is being cleaned (e.g., less abrasive and/or harmful to the enamel of a tooth).

The pH of the oral care compositions disclosed herein may be from about 6 to about 9.5, from about 7 to about 9, or from about 8 to about 9. The pH of the oral care composition may be measured using a pH meter by adding part of the oral care composition into a 30 ml jar, inserting a calibrated pH probe into the jar, and recording the displayed pH values. The pH of the oral care composition may be adjusted with a pH adjusting material such as, without limitations, citric acid, hydrochloric acid, sodium hydroxide, etc.

The pH adjusting agent may be present, individually or in total (if more than one pH adjusting agent is included), in the oral care composition in an amount ranging from about 0.01 wt % to about 2 wt %, from about 0.1 wt % to about 1 wt %, or from about 0.2 wt % to about 0.4 wt % (calculated as the total weight of pH adjusting agent(s) in the oral care composition divided by the total weight of the oral care composition).

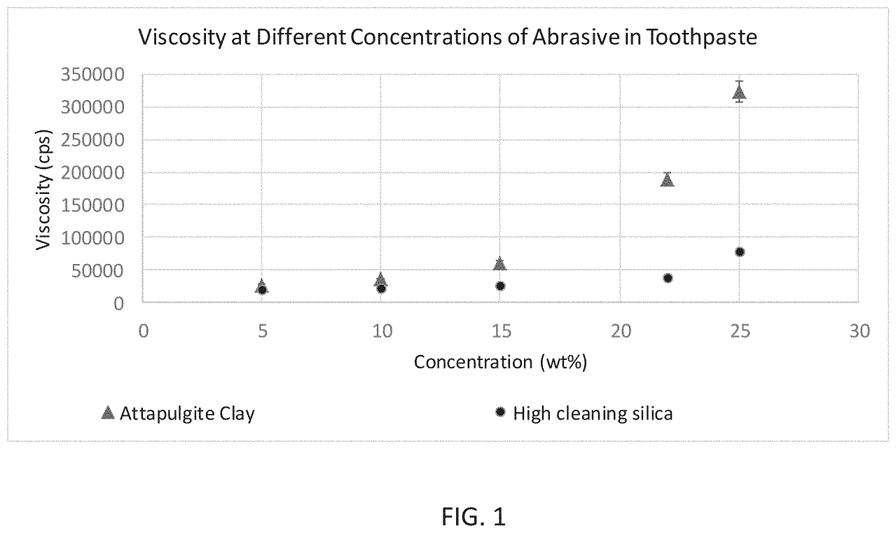

The viscosity of the oral care compositions disclosed herein may be from about 1 cps to about 500,000 cps, from about 25,000 cps to about 350,000 cps, from about 100,000 cps to about 300,000 cps, or from about 150,000 cps to about 275,000 cps. The viscosity value may be calculated as detailed with respect to FIG. 1 in Example 2. An oral care composition with a viscosity value on the lower end of the range may be solution-like or gel-like, whereas an oral care composition with a viscosity value on the upper end of the range may be paste-like.

The oral care compositions disclosed herein may be a dentifrice in a form of toothpaste, gel, mouth wash, powder, mousse, tablet, and the like.

In some embodiments, suitable excipients to be incorporated in the oral care compositions disclosed herein may include, without limitations, flavoring agent(s), active agent(s), colorant(s), dye(s), pigment(s), antioxidant(s), binder(s) (e.g., xanthan gum, polyethylene glycol), solvent(s) (e.g., water, glycerin, sorbitol), humectant(s), viscosity modifier(s) (e.g., block copolymer of propylene oxide and ethylene oxide), foaming agent(s), solubilizer(s), desensitizing agent(s), bleaching agent(s), anti-cavity agent(s) (e.g., sodium F), stain prevention agent(s) (e.g., sodium pyrophosphate), complexing agent(s) (e.g., tetrapotassium pyrophosphate, tetrasodium pyrophosphate), preservative(s) (e.g., sodium benzoate), pH modifying agent(s) (e.g., sodium hydroxide), sweetener(s) (e.g., saccharin), opacifier(s), breath freshening agent(s), soothing agent(s) (e.g., bisabolol), surfactant(s) (e.g., polyglycosides, poloxamers, sodium lauryl sulfate, glutamate), propellant(s) (e.g., propane, isobutane, n-butane, nitrous oxide, and combinations thereof), coupling agent(s), chelating agent(s) (e.g., potassium citrate, sodium pyrophosphate), additional abrasive(s), and combinations thereof. In certain embodiments, the excipient(s) that are included in the oral care compositions disclosed herein are natural so as to minimize irritation and/or allergic reactions to the consumer.

The oral care compositions may include a flavoring agent or a mixture of flavoring agents including natural or synthetic flavorants, such as flavoring oils, flavoring aldehydes, esters, alcohols, similar materials, and combinations thereof. Flavorants may include vanillin, spearmint oil, cinnamon oil, oil of wintergreen (methylsalicylate), peppermint oil, clove oil, anise oil, eucalyptus oil, citrus oils, fruit oils, essences, limonene, menthone, carvone, menthol, anethole, eucalyptol, anethole, eugenol, cassia, oxanone, a-irisone, propenyl guaiethol, thymol, linalool, benzaldehyde, cinnamaldehyde, N-ethyl-p-menthan-3-carboxamine, N,2,3-trimethyl-2-isopropylbutanamide, 3-1-menthoxypropane-1,2-diol, cinnamaldehyde glycerol acetal (CGA), methone glycerol acetal (MGA), cineole, and combinations thereof.

Flavoring agent(s) may be present, individually or in total (if more than one flavoring agent is included), in the oral care composition in an amount ranging from about 0.1 wt % to about 5 wt %, from about 0.2 wt % to about 2 wt %, or from about 0.5 wt % to about 1 wt % (calculated as the total weight of flavoring agent(s) in the oral care composition divided by the total weight of the oral care composition).

The oral care composition may contain a therapeutic active agent in a therapeutically effective amount to treat or prevent an oral condition. Suitable therapeutic active agents may include, without limitations, steroids, NSAIDs, a fluoride ion source (e.g., sodium F anti-cavity agent), polycarboxylate polymers, polyvinyl methyl ether/maleic anhydride (PVME/MA) copolymers, an arginine ester, a zinc ion source, a stannous ion source, delmopinol, tartar control agents, an antibacterial agent, triclosan and salts thereof, chlorhexidine, alexidine, hexetidine, sanguinarine, benzalkonium chloride, salicylanilide, domiphen bromide, cetylpyridinium chloride (CPC), tetradecylpyridinium chloride (TPC), N-tetradecyl-4-ethylpyridinium chloride (TDEPC), octenidine, octapinol, nisin, a zinc ion source, a copper ion source, an essential oil, a furanone, anti-inflammatory agents, antiplaque agents, antioxidants, and a bacteriocins, and salts thereof, honokiol, vitamins, anti-attachment agents, proteinaceous agents, peptides, anesthetics, whitening agents, and combinations thereof.

Active agent(s) may be present, individually or in total (if more than one active agent is included), in the oral care composition in an amount ranging from about 0.1 wt % to about 5 wt %, from about 0.2 wt % to about 2 wt %, from about 0.15 wt % to about 1 wt %, or from about 0.2 wt % to about 0.3 wt % (calculated as the total weight of flavoring agent(s) in the oral care composition divided by the total weight of the oral care composition).

Colorants and/or dyes and/or pigments may be added to the oral care composition in some embodiments. Suitable colorants and/or dyes and/or pigments may include, but not be limited to, colors such as e.g., white, black, yellow, blue, green, pink, red, orange, violet, indigo, brown, and combinations thereof, pigments such as, e.g., Timica Extra Large Sparkles, titanium dioxide and chromium oxide greens, ultramarine blues and pinks and ferric oxides.

Colorant(s) and/or dye(s) and/or pigment(s) may be present, individually or in total (if more than one colorant and/or dye and/or pigment is included), in the oral care composition in an amount ranging from about 0.01 wt % to about 0.5 wt %, from about 0.01 wt % to about 0.2 wt %, from about 0.02 wt % to about 0.4 wt %, or from about 0.03 wt % to about 0.3 wt % (calculated as the total weight of colorant(s) and/or dye(s) and/or pigment(s) in the oral care composition divided by the total weight of the oral care composition).

Antioxidants may be added to the oral care composition in some embodiments. Suitable antioxidants may include, but not be limited to, natural antioxidants such as tocopherol and tocopherol acetate.

The oral care compositions of the invention may contain a binder that could contribute to the rheology of the oral care composition and to the feel of the oral care composition in the oral cavity. Suitable binders include, without limitations, polyvinylpyrrolidone (PVP), marine colloids, carboxyvinyl polymers, starches, cellulosic polymers such as hydroxyethylcellulose, carboxymethylcellulose (carmellose), hydroxypropyl methyl cellulose, hydroxyethylpropylcellulose, hydroxybutyl methyl cellulose, and salts thereof (e.g., carmellose sodium), natural gums such as karaya, xanthan, carrageenans, gellan gum, locust bean gum, gum arabic and tragacanth, chitosan, colloidal magnesium aluminum silicate, colloidal silica, and combinations thereof.

Binder(s) may be present, individually or in total (if more than one binder is included), in the oral care composition in an amount ranging from about 0.1 wt % to about 3 wt %, from about 0.1 wt % to about 2 wt %, from about 0.1 wt % to about 1 wt %, or from about 0.15 wt % to about 1 wt % (calculated as the total weight of binder(s) in the oral care composition divided by the total weight of the oral care composition).

Solvents that may be included in the oral care compositions disclosed herein may include, without limitations, water, alcohols (e.g., isopropyl alcohol), polyhydric alcohols (e.g., glycerin), 1,3-butylene glycol, propylene glycol, hexylene glycol, propane diol, ethylene glycol, diethylene glycol, dipropylene glycol, diglycerin, sorbitol, other sugars which are liquid at room temperature, water soluble alkoxylated nonionic polymers such as polyethylene glycol, and combinations thereof. Solvent(s) may be present, individually or in total (if more than one solvent is included), in the oral care composition in an amount ranging from about 50 wt % to about 95 wt %, from about 60 wt % to about 90 wt %, or from about 65 wt % to about 85 wt % (calculated as the total weight of solvent(s) in the oral care composition divided by the total weight of the oral care composition).

Humectant(s), such as low molecular weight polyethylene glycol (e.g., PEG6-PEG12), may be present, individually or in total (if more than one humectant is included), in the oral care composition in an amount ranging from about 0.1 wt % to about 7 wt %, from about 0.5 wt % to about 7 wt %, or from about 0.5 wt % to about 1 wt % (calculated as the total weight of humectant(s) in the oral care composition divided by the total weight of the oral care composition).

The oral care compositions may include a sweetener capable of providing a palatable and pleasurable factor to the user, and/or capable of masking undesirable flavors present in the dosage form. Exemplary sweeteners that may be in the oral care composition may include, but not be limited to, one or more artificial sweeteners, one or more natural sweeteners, or a combination thereof. Artificial sweeteners include, e.g., acesulfame and its various salts such as the potassium salt (available as Sunett®), alitame, aspartame (available as NutraSweet® and Equal®), salt of aspartame-acesulfame (available as Twinsweet®), neohesperidin dihydrochalcone, naringin dihydrochalcone, dihydrochalcone compounds, neotame, sodium cyclamate, saccharin and its various salts such as the sodium salt (available as Sweet'N Low®), stevia, chloro derivatives of sucrose such as sucralose (available as Kaltame® and Splenda®), and mogrosides. Natural sweeteners include, e.g., glucose, dextrose, invert sugar, fructose, sucrose, glycyrrhizin; monoammonium glycyrrhizinate (sold under the trade name MagnaSweet®); Stevia rebaudiana (Stevioside), natural intensive sweeteners, such as Lo Han Kuo, polyols such as sorbitol, mannitol, xylitol, erythritol, and the like.

Sweetener(s) may be present, individually or in total (if more than one sweetener is included), in the oral care composition in an amount ranging from about 0.05 wt % to about 5 wt %, from about 0.1 wt % to about 1 wt %, or from about 0.1 wt % to about 0.3 wt % (calculated as the total weight of sweetener(s) in the oral care composition divided by the total weight of the oral care composition).

Surfactants may be incorporated in the oral care compositions disclosed herein to aid in the thorough dispersion of the oral care composition throughout the oral cavity and to contribute to cosmetic and foaming properties of the oral care composition. The surfactants that can be included in the oral care composition may be anionic, nonionic, amphoteric compounds, and combinations thereof.

Suitable examples of anionic surfactants are higher alkyl sulfates such as potassium or sodium lauryl sulfate, higher fatty acid monoglyceride monosulfates, such as the salt of the monosulfated monoglyceride of hydrogenated coconut oil fatty acids, alkyl sulfonates such as sodium dodecyl benzene sulfonate, higher fatty sulfoacetates, higher fatty acid esters of 1,2 dihydroxypropane sulfonate, and combinations thereof.

Examples of water soluble nonionic surfactants are condensation products of ethylene oxide with various hydrogen-containing compounds that are reactive therewith and have long hydrophobic chains (e.g., aliphatic chains of about 12 of 20 carbon atoms), which condensation products contain hydrophilic polyoxyethylene moieties, such as condensation products of poly (ethylene oxide) with fatty acids, fatty alcohols, fatty amides and other fatty moieties, and with propylene oxide and polypropylene oxides, e.g., Pluronic materials such as Pluronic F127.

Exemplary suitable alkyl polyglycoside (APG) surfactant(s) that may be used in the oral care compositions disclosed herein may comprise APG C8-C10, APG C10-C16, decyl glucoside, coco-glucoside, anionic APG carboxylate, sodium lauryl glucose carboxylate, lauryl glucoside, D-glucopyranose (oligomeric, C10-16 glycosides, carboxymethyl ethers, sodium salts), C12-C16 fatty alcohol glycoside, and combinations thereof. Exemplary APG surfactant(s) that may be used may have an industry designation of Plantaren® 2000 N UP/MB, Platapon® LGC Sorb, Plantaren® 1200 N UP/MB, and Plantaren® 818 UP/MB.

Surfactant(s) may be present, individually or in total (if more than one surfactant is included) in the oral care composition in an amount ranging from about 0.1 wt % to about 5 wt %, from about 0.3 wt % to about 4 wt %, or from about 0.5 wt % to about 2.5 wt % (calculated as the total weight of surfactant(s) in the oral care composition divided by the total weight of the oral care composition).

In some embodiments, additional abrasive(s) besides magnesium aluminum phyllosilicate may be added to the oral care composition. Suitable abrasive(s) or polishing agent(s) may include, without limitations, silica abrasives such as precipitated silicas, sodium metaphosphate, potassium metaphosphate, tricalcium phosphate, dihydrated dicalcium phosphate, aluminum silicate, calcined alumina, bentonite or other siliceous materials, particulate thermosetting resins, such as melamine, phenolic, and urea-formaldehydes, and cross-linked polyepoxides and polyesters, and combinations thereof.

Abrasive(s) (e.g., magnesium aluminum phyllosilicate and optionally additional abrasive(s)) may be present, individually or in total (if more than one abrasive is included), in the oral care composition in an amount ranging from about 2 wt % to about 35 wt %, from about 2.5 wt % to about 30 wt %, from about 5 wt % to about 25 wt %, from about 10 wt % to about 25 wt %, at about 5 wt %, at about 10 wt %, at about 13 wt %, at about 15 wt %, at about 17 wt %, at about 22 wt %, or at about 25 wt %, or any sub-range or single value therein (calculated as the total weight of abrasive(s) in the oral care composition divided by the total weight of the oral care composition). In some embodiments, the amount of magnesium aluminum phyllosilicate (e.g., attapulgite clay) that is added to the oral care composition is suitable to contribute to the high, yet gentle, cleaning capability of the oral care composition while also providing the desired rheological properties (e.g., viscosity/thickness) to the oral care composition.

The total amount of all excipients in the oral care composition (besides the magnesium aluminum phyllosilicate) may range from about 70 wt % to about 97.5 wt %, from about 75 wt % to about 95 wt %, at about 95 wt %, at about 90 wt %, at about 85 wt %, at about 78 wt %, or at about 75 wt % (calculated as the total weight of all excipients in the oral care composition divided by the total weight of the oral care composition).

In certain embodiments, the oral care composition may include abrasive particulates having a particle size ranging from any of about 5 μm, about 6 μm, about 7 μm, about 8 μm, or about 9 μm to any of about 10 μm, about 11 μm, about 12 μm, about 13 μm, about 14 μm, or about 15 μm.

Some embodiments of the instant disclosure may be directed to a method for preparing any of the oral care compositions disclosed herein. The method may comprise combining the magnesium aluminum phyllosilicate and the excipient(s) to form the oral care composition.

In one embodiment, the oral care composition may be prepared by preparing three separate mixtures. A first mixture may be prepared by mixing a dye, a chelating agent, an antistaining agent, a sweetener, an anti-cavity active ingredient, one or more solvents, and magnesium aluminum phyllosilicate. A second mixture may be prepared by mixing flavor, a humectant/dispersing agent, one or more binders and a solvent. A third mixture may be prepared by mixing one or more surfactants, a pigment, and a solvent. The first mixture may be added to the second mixture and the combined first/second mixture may be homogenized. Subsequently the third mixture may be added to the homogenized combined first/second mixture and the final mixture (first/second/third mixture) may be homogenized.

Some embodiments of the instant disclosure may be directed to a method of cleaning a surface and/or treating a condition in an oral cavity of a subject. The method may comprise contacting the surface in the oral cavity of a subject in need thereof with any of the oral care compositions disclosed herein. If the oral care composition is toothpaste, contacting may encompass brushing the surface in the oral cavity with the oral care composition. If the oral care composition is mouth wash, contacting may encompass rinsing the surface in the oral cavity with the oral care composition.

The surface in the oral cavity of a subject may encompass, for instance, teeth, gums, tongue, cheeks, and combinations thereof.

Some embodiments of the instant disclosure may be directed to a kit comprising any of the oral care compositions disclosed herein enclosed in a suitable container. If the oral care composition is toothpaste, a suitable container may be a toothpaste tube. If the oral care composition is mouth wash, a suitable container may be a mouth wash bottle. If the oral care composition is a mousse, a suitable container may be a sealed pressurizable container. A suitable container may enclose from about 5 ml to about 750 ml, from about 25 ml to about 700 ml, or from about 50 ml to about 650 ml of the oral care composition.

In some embodiments, the kit may further comprise a toothbrush and/or a rinsing cup and/or floss and/or use instructions.

### ILLUSTRATIVE EXAMPLES

The following examples are set forth to assist in understanding the invention and should not be construed as specifically limiting the invention described and claimed herein. Such variations of the invention, including the substitution of all equivalents now known or later developed, which would be within the purview of those skilled in the art, and changes in formulation or minor changes in experimental design, are to be considered to fall within the scope of the invention incorporated herein.

### Example 1: Oral Care Compositions

In this example, five oral care compositions were prepared as summarized in Table 1 below. Oral care compositions 1 through 5 were prepared by mixing the ingredients listed under phase A, while avoiding incorporation of air. Separately, xanthan gum and carrageenan binders from the ingredients of phase B were dispersed in the remaining components of phase B. Phase B mixture was added into phase A mixture and the combined mixture was homogenized. Separately, the polyglycoside and poloxamer surfactants from the ingredients of phase C were dissolved in water. The homogenized mixture of the phase A and phase B ingredients was finally added into the phase C mixture and this final mixture was carefully homogenized.

TABLE 1

| | | | Comp. | Comp. | Comp. | Comp. | Comp. |
|---|---|---|---|---|---|---|---|
| | | | 1 (% | 2 (% | 3 (% | 4 (% | 5 (% |
| Phase | Ingredients | Function | by wt) | by wt) | by wt) | by wt) | by wt) |
| A | Water | Solvent | 11.43 | 10.63 | 9.73 | 8.43 | 7.9 |
| | Sodium Hydroxide | pH adjustor | 0.3 | 0.3 | 0.3 | 0.3 | 0.3 |
| | Glycerin | Solvent | 14 | 12.6 | 12.6 | 12.6 | 12.6 |
| | Sorbitol | Solvent | 50.5 | 47.7 | 43.6 | 37.9 | 35.43 |
| | Puricolor Blue | Dye | 0.04 | 0.04 | 0.04 | 0.04 | 0.04 |
| | Potassium Citrate | Chelating | 1.5 | 1.5 | 1.5 | 1.5 | 1.5 |
| | | agent | | | | | |
| | Sodium | Antistaining | 3 | 3 | 3 | 3 | 3 |
| | Pyrophosphate | agent/chelating | | | | | |
| | | agent | | | | | |
| | Saccharin | Sweetener | 0.15 | 0.15 | 0.15 | 0.15 | 0.15 |
| | Sodium F | Anti-cavity | 0.25 | 0.25 | 0.25 | 0.25 | 0.25 |
| | | agent - active | | | | | |
| | | ingredient | | | | | |
| | Magnesium | Abrasive | 5 | 10 | 15 | 22 | 25 |
| | aluminum | | | | | | |
| | phyllosilicate | | | | | | |
| B | Flavor agent | Flavor agent | 0.8 | 0.8 | 0.8 | 0.8 | 0.8 |
| | Pluracare 600 (PEG | Humectant | 0.7 | 0.7 | 0.7 | 0.7 | 0.7 |
| | 12) | | | | | | |
| | Rheocare XGN | Binder | 0.85 | 0.85 | 0.85 | 0.85 | 0.85 |
| | (xanthan gum) | | | | | | |
| | Carrageenan | Binder | 0.15 | 0.15 | 0.15 | 0.15 | 0.15 |
| | Glycerin | Solvent | 3 | 3 | 3 | 3 | 3 |
| C | Plantacare 1200 UP | Surfactant | 1.5 | 1.5 | 1.5 | 1.5 | 1.5 |
| | (polyglycosides) | | | | | | |
| | Plantaren 818 UP | Surfactant | 0.75 | 0.75 | 0.75 | 0.75 | 0.75 |
| | (poloxamers) | | | | | | |
| | Water | Solvent | 6 | 6 | 6 | 6 | 6 |
| | Timica extra large | Pigment | 0.07 | 0.07 | 0.07 | 0.07 | 0.07 |
| | sparkles | | | | | | |

| | | | | | |
|---|---|---|---|---|---|
| Total | 100 | 100 | 100 | 100 | 100 |
| pH value at 23° C. | 6-9.5 | 6-9.5 | 6-9.5 | 6-9.5 | 6-9.5 |

### Example 2: Viscosity Data

The viscosity at different concentrations of abrasive compound was tested. The abrasive compounds that were tested are attapulgite clay and high cleaning silica. The concentration that were tested are 5 wt %, 10 wt %, 15 wt %, 22 wt %, and 25 wt %, with the wt % being based on a total weight of the sample that was tested. The samples were prepared as described in Example 1 above.

The viscosity was measured with a Brookfield viscometer from Brookfield Engineering Laboratories Inc, with a T-bar spindle (F code 96) at 10 RPM on freshly prepared dentrifices and after three days of preparation (“after +3 days”). The results are summarized in Table 2 below and are depicted in FIG. 1 in a viscosity as a function of abrasive concentration plot. Each data point in the plot of FIG. 1 represents an average (the bars represent a standard deviation) obtained from three viscosity measurements for a particular sample (i.e., three viscosity measurements for each abrasive tested at each of the concentrations tested at each of the time points tested). Viscosity measurements were not taken for the samples with an abrasive concentration of 22 wt % immediately after making. All viscosity measurements, averages, and calculated standard deviations are summarized in Table 2 below in kcps.

TABLE 2
Viscosity of Oral Care Composition with Various Abrasive Concentrations in kcps units

| | Viscosity immediately after making | | | | | | Viscosity after +3 days | | | | | |
|---|---|---|---|---|---|---|---|---|---|---|---|---|
| | | | | High Cleaning | | | | | | High Cleaning | | |
| | Attapulgite Clay | | | Silica | | | Attapulgite Clay | | | Silica | | |
| wt % | | AVG | STD | | AVG | STD | | AVG | STD | | AVG | STD |
| 5 | 25 | 25 | 0 | 15 | 15.83 | 1.44 | 27.5 | 26.67 | 1.44 | 20 | 0 | 0 |
| | 25 | | | 15 | | | 25 | | | 20 | | |
| | 25 | | | 17.5 | | | 27.5 | | | 20 | | |
| 10 | 30 | 29.17 | 1.44 | 15 | 15 | 0 | 37.5 | 37.5 | 0 | 22.5 | 21.67 | 1.44 |
| | 27.5 | | | 15 | | | 37.5 | | | 22.5 | | |
| | 30 | | | 15 | | | 37.5 | | | 20 | | |
| 15 | 45 | 48.33 | 2.89 | 25 | 25 | 07 | 65 | 61.67 | 2.89 | 25 | 25.83 | 1.44 |
| | 50 | | | 25 | | | 60 | | | 27.5 | | |
| | 50 | | | 25 | | | 60 | | | 25 | | |
| 22 | — | — | — | — | — | — | 200 | 190 | 10 | 40 | 38.33 | 2.89 |
| | — | | | — | | | 190 | | | 40 | | |
| | — | | | — | | | 180 | | | 35 | | |
| 25 | 320 | 335 | 30.41 | 75 | 73.33 | 2.89 | 310 | 323.33 | 15.28 | 75 | 78.33 | 2.89 |
| | 315 | | | 70 | | | 340 | | | 80 | | |
| | 370 | | | 75 | | | 320 | | | 80 | | |

As seen in Table 2 and corresponding FIG. 1, the viscosity of the oral care composition increases with increasing concentration of abrasive. An oral care composition with attapulgite clay abrasive exhibits a greater increase in viscosity with increasing attapulgite clay concentration as compared to an oral care composition with high cleaning silica.

### Example 3: Pellicle Cleaning Ratio (PCR) Data

In this example, the PCR values of samples comprising attapulgite clay and high cleaning silica were obtained. The values are summarized in Table 4 below and depicted in FIG. 2.

For the PCR study, three samples were analyzed: 1) an American Dental Association (ADA) reference material, 2) a general abrasive (i.e., attapulgite clay), and 3) a reference abrasive (i.e., high cleaning silica).

### PCR Study—Specimen Preparation

Bovine, permanent, central incisors were cut to obtain labial enamel specimens approximately 9×9 mm² in size. The enamel specimens were then embedded in an auto polymerizing methacrylate resin so that only the enamel surfaces were exposed. The enamel surfaces were then smoothed on a lapidary wheel and polished with flour of pumice and water. Subsequently, the surfaces were sonicated to remove excess debris. The surfaces were then lightly etched (60 seconds in 0.12M HCl [10 ml of 37%/L], 30 seconds in saturated NaCO₃ and 60 seconds in 1.0% phytic acid [2 ml of 50%/L]) to expedite stain accumulation and adherence. The specimen were then placed on a rotating rod alternately exposing them to staining broth containing gastric mucin as a protein source and coffee, tea and FeCl₃.6H₂O as staining sources (i.e., 1.35 g coffee, 1.35 g tea, 0.02 g Fe, and 1.0 g mucin in 400 ml). After about 10 days, the specimens normally develop stain so that the L* value was in the range of 30-38.

### PCR Study—Scoring and Set-Up

The amount of in vitro stain was graded photo-metrically (Minolta 2600d, colorimeter) using only the L* value of the CIELAB scale. Only one batch of specimens from the ones prepared was used. The area of the specimen that was scored was a ¼ inch diameter circle in the center of the enamel sample. Specimens with L* values between 30-38 (30 being more darkly stained) were used. On the basis of these scores, the specimens were balanced into groups of N=16, with each group having the same average baseline score.

The specimens were mounted on a mechanical V-8 cross-brushing machine equipped with soft nylon-filament (Oral-B 40 Indicator) toothbrushes. Toothbrushes were conditioned by running the brushing machine for 1,000 strokes in deionized water on non-usable specimens. Tension on the enamel surface was adjusted to 150 g. The powders were used as slurries prepared by mixing (10 g of powder with 50 ml of 0.5% CMC solution (carboxymethyl cellulose/glycerin)). The ADA reference material was prepared by mixing 10 g of calcium pyrophosphate reference material and 50 ml of a 0.5% CMC solution. The specimens were brushed for 800 double strokes. To minimize mechanical variables, one specimen per group was brushed on each of the eight brushing heads. Fresh slurries were used for each specimen brushed. Following brushing, specimens were rinsed, blotted dry, and scored again for stain as previously described.

### PCR Study—Calculations

The difference between the pre- and post-brushing L* values was determined and the Mean (N=16) standard deviation (SD) and standard error (SEM) calculated.

The cleaning ratio for the reference material group was assigned a value of 100. The mean decrement for the reference group was divided into 100 to obtain a constant value to multiply times each individual test decrement within the study. The individual cleaning ratio of each specimen was then calculated (decrement×constant). The Mean (N=16), SD and SEM for each group (N=16) was then calculated using the individual cleaning ratios. The larger the value of the cleaning ratio, the greater the amount of stained pellicle removed in this test.

### Example 4: Relative Dentin Abrasivity (RDA) Data

In this example, the RDA values of samples comprising attapulgite clay and high cleaning silica were obtained. The values are summarized in Table 4 below and depicted in FIG. 2. The purpose of this study was to determine the relative abrasion level of dentifrices to dentin.

### RDA Study—Procedure

The procedure used was the ADA recommended procedure for determination of dentifrice abrasivity (ADA/ANSI No. 130 and ISO 11609). The dentin specimens (8) were placed in a neutron flux under the controlled conditions outlined by the ISO/ADA. The specimens were then mounted in methyl-methacrylate to fit in a V-8 cross-brushing machine. The specimens were brushed for a 1500/5000 (depending on the condition of the specimens according to SOP Tm118.01) stroke, precondition run using a slurry consisting of 10 g ISO/ADA reference material (calcium pyrophosphate) in 50 ml of a 0.5% CMC (carboxymethyl cellulose/glycerin solution). The brushes used were those specified by the ADA and ISO, and brush tension was 150 g.

Following the precondition run, the test was performed using 150 g and 1500 strokes in a sandwich design in which each test material slurry (10 g powdered abrasive material in a 50 ml solution of 0.5% CMC/glycerin) was flanked by the reference material slurries (10 g ISO/ADA reference material in a 50 ml 0.5% CMC/glycerin solution) as outlined in Table 3 below:

TABLE 3

| Run | Treatment |
|---|---|
| 1 | ADA Reference Material |
| 2 | Sample 1: General Abrasive (i.e., attapulgite clay) |
| 3 | ADA Reference Material |
| 4 | Sample 2: Reference Abrasive (i.e., high cleaning silica) |
| 5 | ADA Reference Material |

One (1.0) ml samples were taken, weighed (0.01 g), and added to 4.5 ml of scintillation cocktail. The samples were then mixed well and immediately put on a liquid scintillation counter for radiation detection. Following counting, the net counts per minute (CPM) values were divided by the weight of the sample to calculate a net CPM/gram of slurry. The net CPM/g of the pre and post ADA reference material for each test slurry were then calculated and averaged to use in the calculation of RDA (relative dentin abrasion) for the test material. The ISO/ADA material was assigned a value of 100 and its ratio to the test material calculated*. *As of 8-16-99, a new ADA reference material had been used. This material is 3.6% less abrasive than old batches, thus requiring the addition of 3.6% to the reference material CPM when comparing the data disclosed herein to data predating 8-16-99.

TABLE 4
PCR and RDA Values for Abrasive Samples

| | PCR | PCR STD Err | RDA | RDA STD Err |
|---|---|---|---|---|
| Attapulgite clay | 81 | 4 | 46 | 3 |
| High cleaning silica | 69 | 3 | 78 | 2 |
| ADA Reference | 100 | 3 | — | — |

The PCR and RDA values were obtained from abrasive samples prepared as described above and are representative of the performance of the various abrasives when they are incorporated into an oral care composition. The measured PCR to RDA ratio of the high cleaning silica was about 0.875. In comparison the measured PCR to RDA ratio of the attapulgite clay was about 1.8 indicating that the attapulgite clay exhibits a better pellicle cleaning ratio and a lower relative dentin abrasivity as compared to high cleaning silica.

### Example 5: Relative Dentin Abrasivity (RDA) Values Across a Range of Abrasive Concentrations

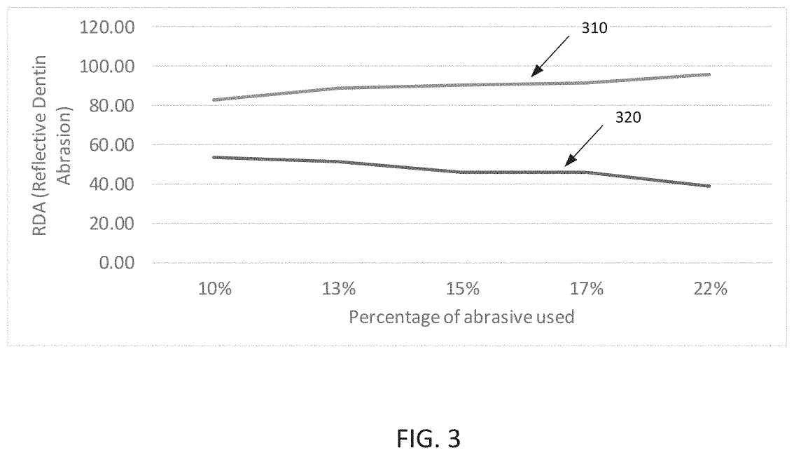

In this example, the RDA values of samples comprising a range of abrasive concentrations were obtained. The abrasives that were tested were attapulgite clay and high cleaning silica. The RDA values are summarized in Table 5 below and depicted in FIG. 3. As can be seen from Table 5 and from FIG. 3, attapulgite clay (depicted as line 320) has lower RDA values across all tested concentration as compared to the RDA values of high cleaning silica (depicted as line 310). The lower RDA values for the attapulgite clay being indicative of it being more gentle and less abrasive towards the enamel as compared to high cleaning silica. This was shown across all of the abrasive concentrations that were tested.

Furthermore, from line 320, it can be seen that a higher concentration of attapulgite clay exhibited lower RDA values. The abrasivity of the attapulgite clay trended downwards with increasing concentrations of attapulgite clay. This was depicted through the decreasing RDA values with increasing concentrations of abrasive, shown via line 320. In contrast, the high cleaning silica abrasive exhibited the opposite trend, increasing RDA values with increasing abrasive concentration, shown via line 310.

The RDA values of these samples were determined in a similar manner as the RDA values in Table 4.

TABLE 5
RDA Values for Abrasive Samples

Abrasive Concentration (wt %)

| | 10 | 13 | 15 | 17 | 22 |
|---|---|---|---|---|---|
| Abrasive Type | wt % | wt % | wt % | wt % | wt % |
| Attapulgite clay | 53.67 | 51.01 | 46.00 | 46.10 | 38.9 |
| High cleaning silica | 82.55 | 88.44 | 90.20 | 91.50 | 95.71 |

### Example 6: Pellicle Cleaning Ratio (PCR) Values Across a Range of Abrasive Concentrations

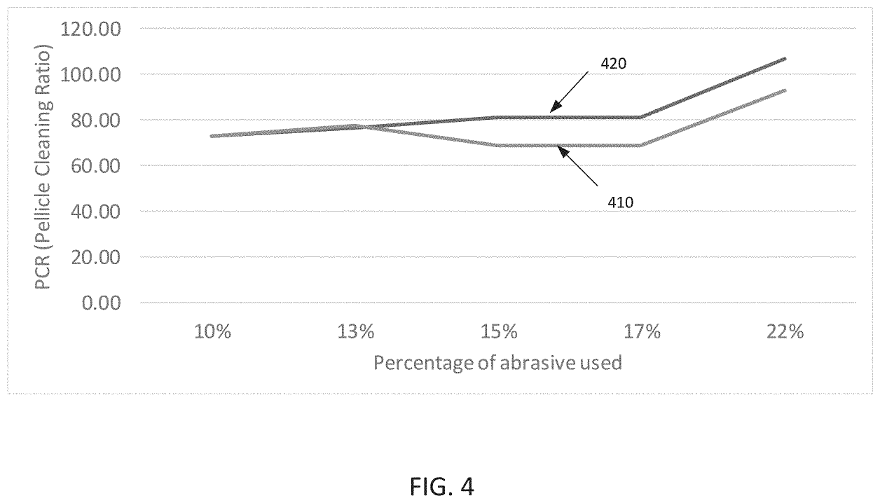

In this example, the PCR values of samples comprising a range of abrasive concentrations were obtained. The abrasives that were tested were attapulgite clay and high cleaning silica. The PCR values are summarized in Table 6 below and depicted in FIG. 4. As can be seen from Table 6 and from FIG. 4, attapulgite clay (depicted as line 420) has comparable or higher PCR values across all tested concentration as compared to the PCR values of high cleaning silica (depicted as line 410). Higher PCR values being indicative of the cleaning being more effective. The attapulgite clay exhibited similar cleaning efficacy to that of high cleaning silica at lower concentration (i.e., similar PCR values at the 10 wt % and 13 wt % data points). At higher concentrations, the attapulgiteclay exhibited better cleaning efficacy as compared to that of high cleaning silica (i.e., higher PCR values for the attapulgite clay at the 15 wt %, 17 wt %, and 22 wt % data points).

The PCR values of these samples were determined in a similar manner as the PCR values in Table 4.

TABLE 6
PCR Values for Abrasive Samples

Abrasive Concentration (wt %)

| | 10 | 13 | 15 | 17 | 22 |
|---|---|---|---|---|---|
| Abrasive Type | wt % | wt % | wt % | wt % | wt % |
| Attapulgite clay | 73.10 | 76.70 | 81.00 | 81.10 | 107 |
| High cleaning silica | 72.90 | 77.60 | 69.00 | 68.80 | 93 |

### Example 7: Particle Size Analysis

Powder samples of attapulgite clay were analyzed for particle size using Malvem Mastersizer® 3000 Particle Size Analyzer* (Malvem Instruments, Southborough, Mass.) in dry powder mode (Analysis 1). Results were reported in terms of a volume-weighted mean diameter D[4,3], and the diameter at which 10%, 50%, and 90% of the population is smaller was given as d(0.1), d(0.5), and d(0.9). Surface statistics were based on estimated particle density of 2.0 g/cm³. *The Mastersizer® 3000 uses laser diffraction, also called Mie scattering to measure particles in the range of 0.01-3000 pin. In dry powder mode, the instrument uses a single source: a red Helium Neon Laser to measure forward scattering, side scattering, and backscattered signals. The sample is dispersed using an Aero S dry powder dispersing unit and operations are performed using a standard operating procedure (SOP) created specifically to include such sample parameters as refractive index (e.g., attapulgite clay particles refractive index of 1.68 with a particle absorption index of 0.10, and air dispersant refractive index of 1.00 or IPA dispersant refractive index of 1.390), air pressure (e.g., 1.6 bar), analysis time, and number of measurements (e.g., about 90 measurements). For the wet measurement, the following should be noted: The instrument uses two light sources: a red Helium Neon Laser to measure forward scattering, side scattering, and backscattered signals, and a blue monochromatic light source to measure wide angle forward and back scattered signals. The sample is dispersed using a Hydro MV medium volume recirculator.

To assess the level of hard agglomerates, the sample was also run after dispersing in isopropyl alcohol (IPA) (Analysis 2) and analysis without sonication was compared to results after 5 minutes' sonication (Analysis 3) and 10 minutes' sonication (Analysis 4).

The results for Analysis 1, 2, 3, and 4 are summarized in Tables 7 and 8 below.

TABLE 7
Particle Size Analysis Results

| | D[4,3] | d(0.1) | d(0.5) | d(0.9) |
|---|---|---|---|---|
| | (μm) | (μm) | (μm) | (μm) |
| Analysis 1 - Dry | 12.7 | 2.31 | 9.68 | 26.2 |
| Analysis 2 - Wet | 14.7 | 4.03 | 11.9 | 29.5 |
| (IPA, no sonication) | | | | |
| Analysis 3 - Wet | 13.6 | 3.92 | 11.3 | 26.8 |
| (IPA, 5 min sonication) | | | | |
| Analysis 4 - Wet | 13.5 | 3.90 | 11.3 | 26.5 |
| (IPA, 10 min sonication) | | | | |

TABLE 8
Particle Size Analysis Results

| | | | | | Surface | Specific |
|---|---|---|---|---|---|---|
| | | | | | Weighted | Surface |
| | | Weighted | | | Mean D[3,2] | Area |
| | Obscuration | Residual | Uniformity | Span | (μm) | (m²/kg) |
| Analysis 1 - | 2.09% | 0.19% | 0.798 | 2.466 | 5.08 | 590.6 |
| Dry | | | | | | |
| Analysis 2 - | 7.45% | 0.58% | 0.663 | 2.144 | 8.47 | 354.2 |
| Wet (IPA, no | | | | | | |
| sonication) | | | | | | |
| Analysis 3 - | 6.89% | 0.73% | 0.623 | 2.018 | 8.15 | 368.3 |
| Wet (IPA, 5 | | | | | | |
| min sonication) | | | | | | |
| Analysis 4 - | 6.65% | 0.64% | 0.620 | 2.005 | 8.10 | 370.2 |
| Wet (IPA, 10 | | | | | | |
| min sonication) | | | | | | |

As can be seen from Table 7, the particle size distribution (PSD) of the attapulgite clay remained substantially unchanged or changed slightly when comparing the PSD of the dry sample (Analysis 1) to the PSD of the wet sample (Analysis 2). This suggests that the particle size of the attapulgite clay particles remains substantially the same upon liquid introduction.

Furthermore, as can be seen from Table 7, the PSD of the attapulgite clay remained substantially unchanged or changed slightly when comparing the PSD of the wet sample without sonication (Analysis 2) to the wet sample with sonication (Analysis 3 and Analysis 4). This suggests that the samples contained substantially no agglomerates or a low level of agglomerates that were dispersed via sonication.

Hence, it is believed, without being construed as limiting, that the particle size of the attapulgite clay particles will remain substantially the same and will not aggregate when the abrasive is formulated.

For simplicity of explanation, the embodiments of the methods of this disclosure are depicted and described as a series of acts. However, acts in accordance with this disclosure can occur in various orders and/or concurrently, and with other acts not presented and described herein. Furthermore, not all illustrated acts may be required to implement the methods in accordance with the disclosed subject matter. In addition, those skilled in the art will understand and appreciate that the methods could alternatively be represented as a series of interrelated states via a state diagram or events.

In the foregoing description, numerous specific details are set forth, such as specific materials, dimensions, processes parameters, etc., to provide a thorough understanding of the present invention. The particular features, structures, materials, or characteristics may be combined in any suitable manner in one or more embodiments. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. Reference throughout this specification to “an embodiment”, “certain embodiments”, or “one embodiment” means that a particular feature, structure, or characteristic described in connection with the embodiment is included in at least one embodiment. Thus, the appearances of the phrase “an embodiment”, “certain embodiments”, or “one embodiment” in various places throughout this specification are not necessarily all referring to the same embodiment.

The present invention has been described with reference to specific exemplary embodiments thereof. The specification and drawings are, accordingly, to be regarded in an illustrative rather than a restrictive sense. Various modifications of the invention in addition to those shown and described herein will become apparent to those skilled in the art and are intended to fall within the scope of the appended claims.

1. An oral care composition comprising a magnesium aluminum phyllosilicate compound and an excipient, the magnesium aluminum phyllosilicate having a (pellicle cleaning ratio):(relative dentin abrasivity) (PCR:RDA) ratio ranging from about 0.9 to about 10.

2. An oral care composition comprising a magnesium aluminum phyllosilicate compound and an excipient, the oral care composition having a pH ranging from about 6 to about 9.5.

3. An oral care composition comprising a magnesium aluminum phyllosilicate compound and an excipient, the oral care composition having a viscosity ranging from about 25,000 cps to about 350,000 cps.

4. The oral care composition of claim 1, wherein the magnesium aluminum phyllosilicate compound is present at a concentration of from about 2.5 wt % to about 30 wt %, or from about 5 wt % to about 25 wt %, based on total weight of the oral care composition.

5. The oral care composition of claim 1, wherein the oral care composition has a pH of from about 6 to about 9.5, or from about 7 to about 9, or from about 8 to about 9.

6. (canceled)

7. (canceled)

8. The oral care composition of claim 1, wherein the PCR:RDA ratio ranges from about 1.2 to about 5, or from about 1.5 to about 3.

9. The oral care composition of claim 1, wherein the oral care composition has a viscosity ranging from about 25,000 cps to about 350,000 cps, from about 100,000 cps to about 300,000 cps or from about 150,000 cps to about 275,000 cps.

10. (canceled)

11. The oral care composition of claim 1, wherein the oral care composition is a toothpaste, a gel, a mouth wash, a mousse, a powder, or a tablet.

12. The oral care composition of claim 1, wherein the magnesium aluminum phyllosilicate has the formula (Mg,Al)₂Si₄O₁₀(OH).4(H₂O).

13. The oral care composition of claim 1, wherein the magnesium aluminum phyllosilicate is an attapulgite clay.

14. The oral care composition of claim 1, wherein the magnesium aluminum phyllosilicate is a composite of smectite and polygorskite.

15. The oral care composition of claim 1, wherein the magnesium aluminum phyllosilicate has a PCR value of from about 60 to about 100, from about 65 to about 95, or from about 70 to about 90.

16. The oral care composition of claim 1, wherein the o magnesium aluminum phyllosilicate has a RDA value of from about 20 to about 70, from about 25 to about 65, or from about 35 to about 55.

17. The oral care composition of claim 1, wherein the excipient comprises one or more of flavoring agent(s), active agent(s), colorant(s), dye(s), pigment(s), antioxidant(s), binder(s), solvent(s), humectant(s), viscosity modifier(s), foaming agent(s), solubilizer(s), desensitizing agent(s), bleaching agent(s), anti-cavity agent(s), stain prevention agent(s), complexing agent(s), preservative(s), pH adjusting agent(s), sweetener(s), opacifier(s), breath freshening agent(s), soothing agent(s), surfactant(s), propellant(s), coupling agent(s), chelating agent(s), additional abrasive(s), and combinations thereof.

18. The oral care composition of claim 17, wherein the solvent comprises one or more of water, glycerin, sorbitol, and combinations thereof.

19. The oral care composition of claim 17, wherein the excipient comprises a solvent at a concentration of from about 60 wt % to about 90 wt %, or from about 65 wt % to about 85 wt %, based on total weight of the oral care composition.

20. The oral care composition of claim 17, wherein the excipient comprises one or more of:
a dye at a concentration of from about 0.01 wt % to about 0.2 wt %;
a sweetener at a concentration of from about 0.1 wt % to about 1 wt %;
a flavoring agent at a concentration of from about 0.2 wt % to about 2 wt %;
a humectant at a concentration of from about 0.5 wt % to about 7 wt %;
a binder at a concentration of from about 0.1 wt % to about 2 wt %; or
combinations thereof,
wherein all concentrations are based on a total weight of the oral care composition.

21. A method for preparing the oral care composition of claim 1, the method comprising combining the magnesium aluminum phyllosilicate and excipient.

22. A method for cleaning a surface in an oral cavity of a subject or for treating a condition in an oral cavity of a subject, the method comprising contacting the surface in the oral cavity of a subject in need thereof with the oral care composition of claim 1.

23. (canceled)

24. (canceled)

25. (canceled)

26. A kit comprising: the oral care composition of claim 1 enclosed in a container.

27. (canceled)

28. (canceled)
